(19) United States
(12) Patent Application Publication
(10) Pub. No.: US 2025/0349426 A1
(43) Pub. Date: Nov. 13, 2025

(54) HYPER-PERSONALIZED TREATMENT BASED ON CORONARY MOTION FIELDS AND BIG DATA

(71) Applicant: KONINKLIJKE PHILIPS N.V., EINDHOVEN (NL)

(72) Inventors: DANIËL SIMON ANNA RUIJTERS, EINDHOVEN (NL); PAULA ANDREA RUDENICK, EINDHOVEN (NL)

(21) Appl. No.: 18/875,414

(22) PCT Filed: Jun. 20, 2023
(86) PCT No.: PCT/EP2023/066519

§ 371 Date: Dec. 16, 2024

Related U.S. Application Data
(60) Provisional application No. 63/357,020, filed on Jun. 30, 2022.

(51) Int. Cl. G16H 50/20; G16H 10/60; G16H 50/70

(52) U.S. Cl. CPC G16H 50/20; G16H 10/60; G16H 50/70

(57) ABSTRACT

System (SYS) and related method for predicting a patient treatment option. The system may comprise an input interface (IN) for receiving input data including biodynamical measurements in respect of a patient. A predictor module (PM) configured to process the biodynamical measurements to obtain output data including an indication for a treatment option for the patient.

### FIELD OF THE INVENTION

The invention relates to a system for predicting a patient treatment option, a training system for training such a system, related methods, a computer program element, and a computer readable medium.

### BACKGROUND OF THE INVENTION

Personalized medicine (or “precision medicine”), is a medical approach that that some have defined as “ . . . tailoring of medical treatment to the individual characteristics of each patient. It does not literally mean the creation of drugs or medical devices that are unique to a patient but rather the ability to classify individuals into subpopulations that differ in their susceptibility to a particular disease or their response to a specific treatment. Preventive or therapeutic interventions can then be concentrated on those who will benefit. sparing expense and side effects for those who will not”. See J Watkins et al in “Therapeutic Delivery”, published November 1(5), pp 651-65, 2010. citing a US government report.

Modern guidelines on clinical treatment are usually based on cohort studies, taking broad patient characteristics into account.

However, due to the static nature of the guidelines and associated decision mechanisms, there are limitations to the number of parameters and decision points that can be taken into account in such guidelines or current efforts at personalized medicine.

### SUMMARY OF THE INVENTION

There may therefore be a need for a technical solution for improving medical practice. An object of the present invention is achieved by the subject matter of the independent claims where further embodiments are incorporated in the dependent claims. It should be noted that the following described aspect of the invention equally applies to the related methods, to the training system, to the computer program element and to the computer readable medium.

According to a first aspect of the invention there is provided a system for predicting a patient treatment option, comprising:
input interface for receiving input data including biodynamical measurements in respect of a patient (to be treated);
a predictor module configured to process the biodynamical measurements to obtain output data including an indication for a treatment option for the said patient.

In embodiments, the biodynamical measurements is a time series.

In embodiments, the predictor module is preferably based on a trained machine learning model, previously trained on patient data from a cohort of patients.

In embodiments, the machine learning model is implemented as, or is based on, any one of: i) a clustering algorithm, ii) one or more artificial neural networks, iii) a support vector machine.

Implementation based on a clustering algorithm is preferred. The training data may be clustered into clusters.

In embodiments, the different treatment options correspond to different clusters, and wherein the indication includes an indication of one or more of the clusters.

In embodiments, the clustering algorithm is based on a similarity measure, based on which similarity measure the indication is provided. The similarity measure may relate to a similarity between the measurement data for two given patients. The similarity may be used in training phase to divide the training data based on a patient cohort into distinct clusters. After training, in deployment or testing, the treatment option prediction for a given patient is computed based on the similarity measure or a related measure. Specifically, a similarity between the given patient and the predefined clusters is established using the similarity measure. Conceptually, the similarity measure may also be understood as distance measure in some embodiments. The given patient may fall into more than one cluster. Using a clustering algorithm thus avoid “hard” classification and takes better into account that a given patient may benefit from more than one treatment option or from a mix of such options. Whilst clustering type algorithms have been found to be of particular benefit herein, other machine learning model approaches such as ii), iii) listed above or not excluded herein and still may be used, either in combination with clustering or instead thereof. Fuzzified classification models and/or or multi-class classification models may be used in some embodiments.

In embodiments, the system includes a graphics display generator configured to generate a graphics display for display on a display device, the graphics display to visualize the said indication.

In embodiments, the graphics display including a visualization of the clusters and a graphical indicator in respect of the patient indicative of similarity or proximity to the said clusters. Visualization of the predicted treatment option indicator against the pre-defined clusters as per the training data allows user to quickly ascertain suitable treatment options, in particular for patients with mixed needs.

In embodiments, the biodynamical measurements incudes any one of: coronary vessel motion data, perfusion data, ECG (electrocardiogram) data, EEG (electroencephalogram) data, oxygenation data.

Coronary vessel motion data have been found to be a particularly robust (bio-)marker/indicator for treatment options, in particular, but not only, in relation to the heart, such as types of stenosis treatments, etc, or for other heart treatments, and treatments beyond the heart. Coronary vessel motion data may be represented by motion fields, in 4D (3D+t) or 2D+1, such as based on segmented cardiac imagery. Motion field may be based on registration of over time frames. Coronary vessel motion data may be prepared and used as descriptive vector or matrix representative of such motion fields.

In embodiments, the biodynamical measurements include image data in projection or image domain.

In embodiments, the coronary vessel data is based on back-projected vessel segmentations in multi-directional projection imagery acquired of the patient.

In embodiments, the coronary vessel data is based on registration of a series of 2D projection images.

In embodiments, the output data includes outcome data for the treatment option. In another aspect there is provided a training system for training, based on the training data, the machine learning model of the system of any one of the previous claims.

In embodiments, the training system is implemented based on a clustering algorithm and a similarity measure, wherein the training system is to define clusters in the training data based on the said similarity measure.

In another aspect there is provided a computer-implemented method for predicting a patient treatment option, comprising:
receiving input data including biodynamical measurements in respect of a patient: and
processing the biodynamical measurements to obtain output data including an indication for a treatment option for the said patient.

In another aspect there is provided a computer-implemented method for training, based on training data, the machine learning model of the system any one of the previous claims 1-13.

In another aspect there is provided at least one computer program element, which, when being executed by at least one processing unit or system, is adapted to cause the processing unit to perform the method as per any one of the above mentioned embodiments.

In another aspect there is provided at least one computer readable medium having stored thereon the program element or having stored thereon the machine learning model as in sued in the system of any one of the above mentioned embodiments.

In another aspect there is provided a use of the trained machine learning model for predicting treatment option for a given patient.

The proposed system or method allow practicing personalized medicine by taking, if required, a large number of characteristics of the current patient into account. The system affords a technical solution to aid medical decision making. Costs can be driven down, patient comfort increased, and wear-and-tear effects on expensive medical devices due to unnecessary use can be reduced. In X-ray equipment for example, anode disks are subjected to very high temperature gradients. If the X-ray equipment is used in a wrong manner, such as unnecessary use, premature degradation may result as a consequence of choosing the wrong/not needed treatment option for the patient. And, even more worrying, the patient is deprived from the treatment he or she did need. The proposed system allows hyper-personalized approach. in that patients can benefit from tailored “translation” of cohort data to their specific characteristics. The cohort data may comprise large amounts of data. in many dimensions. The biodynamical measurement may be represented as a potentially high dimensional descriptive vector or matrix/tensor. thus increasing expressiveness. The descriptive vector may be used as look-up key in searching for treatment options for a given patient, such database look-up operations being contemplated herein in some embodiments.

The predictor module may perform in some embodiments a lookup operation in a patient database, in order to arrive at a much more personalized analysis of the available data in taking a potentially large of number of patient characteristics parameters into account. This hyper-personalization may lead to better tailored outcome predictions, and therefore aids in establishing an optimal treatment plan for example. In some preferred embodiments, machine learning (“ML”) approaches are used to train an ML model on large amounts of patient data held in database(s), the predictor module using such a trained model to predict, that is compute, the treatment option. Predictor module may preferably operate in in real-time.

In some embodiments. the biodynamical measurements may be represented as a descriptive vector that describes motion of anatomical patient features, such as in particular motion of coronary arteries. Patients with coronary artery disease have lesions in their coronaries, typically stenosis, which may lead to impaired heart function. The coronaries' function is to provide oxygenated blood to the heart muscle, and this function is sadly impaired in such patients. Stenosis can be a single. focal lesion, or can be severe and multiple stenoses can be present in multiple vessels. The stenosis' degree can vary, and it can be building up for many years or be acute. All these factors and others impact the severity of the impairment of the heart muscle. and. as a consequence. leads to different motion patterns it has been found herein. Such motion pattern. furthermore. can express aspects that cannot be evaluated when simply measuring the stenosis degree, such as the duration of the disease, and the viability and vitality of the muscle. It appears these factors do contribute to the possible treatment outcome and treatment options.

In some embodiments as proposed herein, coronary motion patterns are determined based on a preferably large pool of prior data as may be found in current (large) medical database of coronary disease patients. The patterns may be clustered into descriptive clusters. A correlation between motion pattern and disease progression. optimal treatment decisions. optimal follow-up regime. risk factors, etc., can be determined. Since the motion pattern have been found to be representative of disease severity, location, and multiplicity, a correlation with the aforementioned factors may be expected. The clustering is performed by using a suitable similarity measure to compare motion descriptive vectors.

Then, once the clustering is done, and a (new) particular patient is to be diagnosed or treated, the motion pattern for that patient can be established. Consequently, the cluster (and sub-cluster) the patient falls into can be established by comparing the patient's motion pattern to the motion patterns in the clusters according to the similarity measure. Since the cluster is expressive for patient outcome and optimal treatment options, this information can be used to determine the optimal treatment strategy. perform device selection, establish a follow-up plan and risk monitoring plan.

Motion patterns of cardiac vessels are however one example for biodynamical measurements, and other examples, such as perfusion, EEG, ECG, etc, are also envisaged herein. Thus, biodynamical measurements are not necessarily related to physical motion of one or more anatomical features, but may relate to other biological quantities and their over-time evolution. Clustering approaches are preferred herein as clustering can account well for capturing the notion of treatment options at the right level of fuzziness.

“User” relates to a person, such as medical personnel or other, operating an imaging apparatus, or overseeing or managing at least in parts, a medical procedure in relation to a patient. In other words, the user is in general not the patient, however it is not excluded herein that in some applications it is the patient who may use the system as proposed herein, for example for information purposes.

“Biodynamical measurement(s)” may not necessarily relate to raw data, but may relate to such raw data or derived data, possibly prepared to foster efficient processing, such as scaling or other type of mapping. Biodynamical measurement(s) may be represented as one or more time series or sequence(s) of data measurement values, and may be referred to herein in the singular or plural. Biodynamical measurement(s) should be construed broadly to include any time varying measurements acquired in one single measurement session or over multiple such sessions, possibly interrupted by one or more time gap(s). In such one or more sessions, a sensor/transducer, suitable, preferably intended for medical purpose, is applied to the patient to acquire the biodynamical measurement(s). Biodynamical measurements may vary in space and time, such as sequences of medical image data. The data items may be scalar, or may be higher dimensional such as 2D, or 3D or 4D or of higher dimensional still. The Biodynamical measurement(s) may be acquired by suitable sensor(s) of a patient, and may represent a time evolution of one or more medical, anatomical features, or biological/biomedical over time quantity. Biodynamical measurement(s) may relate to motion, deformations, etc., or other changes in relation to one or more organs, one or more tissues, or one or more anatomical features. Biodynamical measurement(s) may be relatable to treatment options and optionally, to outcome data.

In general, the term “machine learning” includes a computerized arrangement (also 30 referred to herein a “module”) that implements a machine learning (“ML”) algorithm. Some such ML algorithms operate to adjust a machine learning model that is configured to perform (“learn”) a task. This adjusting or updating of model is called “training”. In some applications (implicit modeling), it is the training data that is, or is part of, the model. In such implicit setups, the adjusting of the model may broadly include structuring the training data, such as in clustering. In general task performance by the ML module may improve measurably, with training experience. Training experience may include suitable training data and exposure of the model to such data. Task performance may improve the better the data represents the task to be learned. “Training experience helps improve performance if the training data well represents a distribution of examples over which the final system performance is measured”. The performance may be measured by objective tests based on output produced by the module in response to feeding the module with test data. The performance may be defined in terms of a certain error rate to be achieved for the given test data. See for example, T. M. Mitchell, “Machine Learning”, page 2, section 1.1, page 6, section 1.2.1, McGraw-Hill, 1997.

### BRIEF DESCRIPTION OF THE DRAWINGS

Exemplary embodiments of the invention will now be described with reference to the following drawings, which, unless stated otherwise, are not to scale, wherein:

FIG. 1 shows a computer implemented medical arrangement for computing treatment options for a given patient;

FIG. 2 shows a graphics display generated by the system of FIG. 1 in embodiments;

FIG. 3 shows a representation of medical image data as examples of biodynamical measurements as may be used herein by the system in some embodiments;

FIG. 4 shows a machine learning model as may be used in embodiments;

FIG. 5 shows a block diagram of a training system for training a machine learning model as may be used in embodiments herein;

FIG. 6 shows a computer-implement method of computing treatment options for a given patient based on biodynamical measurements; and

FIG. 7 shows a flow chart of a method of training a machine learning model based on training data.

### DETAILED DESCRIPTION OF EMBODIMENTS

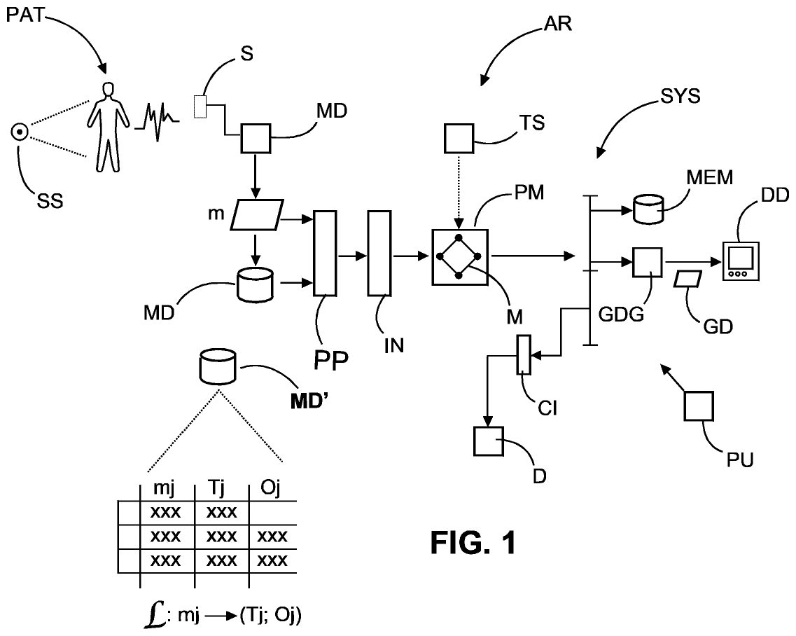

Reference is now made to the block diagram of FIG. 1 which shows a medical arrangement AR. As will be explained in more detail below, the arrangement AR is capable of generating treatment option(s) T for a patient, optionally with associated outcome data o, based on medical measurement data (“medical measurements”) for the given patient PAT.

The arrangement AR includes in some embodiments, but not necessarily in all embodiments, a medical measuring device MD. The medical measuring device MD may use one or more sensors S to acquire the medical measurements for the given patient PAT. The measurements m may be supplied to a measurement processing system SYS that is configured to produce (for example, compute) the treatment option T for the given patient PAT based on the supplied measurements. A said, the outcome data o associated with the treatment option T is optional and the following will refer simply to the treatment option T as output provided by the system SYS, with the understanding that this output may or may not include the associated outcome data o.

The medical measurements m as used herein refer to biodynamical measurements (data) m. The biodynamical measurements m may be received by system SYS in an online embodiment via a communication channel from a measurement device MD that is operable to acquired such measurements in relation to a given patient PAT. Alternatively, or in addition, the measurements m may be received in an off-line embodiment from a data storage MD, such as a medical database or other where previous measurements of the given patient are stored. Thus, the biodynamical measurements may be stored after acquisition in storage MD first, and may then by retrieved for processing by system SYS on demand, for example on query by a medical user.

In either case, the one or more biodynamical measurements m are received at data input port IN of the system SYS and are processed by a predictor module PM of the system SYS. The predictor module PM produces output data based on the biodynamical measurements m so received. The output data may include an indication of the associated treatment option T based on the one or more biodynamical measurements m. The output data including the computed/predicted treatment option may be stored in a data storage, such as in the said medical database MD, or may be stored elsewhere. The computed treatment option T may be stored as an entry of the patient's health record. Any one or more of a manifold of uses of the output data are envisaged herein. For example, the output data may be displayed on a display device DD, or may be otherwise processed, such as by a medical data analytics system, or may be used to control a medical device D to effect treatment of patient PAT, etc.

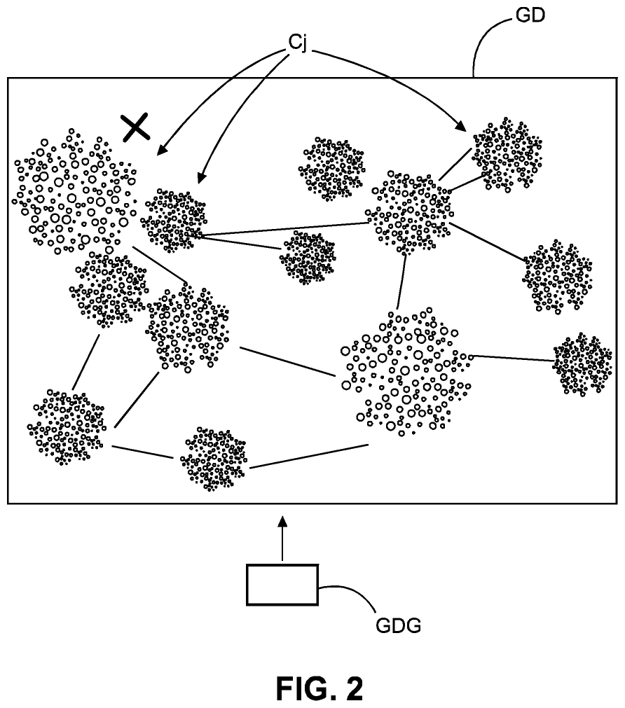

The output data including treatment option T may be provided in a suitable form, such as text data (natural language or coded), numerical data, graphical data in form of a graphics display, or as a combination of two or more of the foregoing. Specifically, a graphical display generator GDG may be operative to generate the graphics display GD. The graphics display GD may be displayed on a display device DD for example. An example of this graphics display GD including an indication of the (one or more) treatment options T is shown in FIG. 2, which will be explained in more detail below. In addition or instead of so displaying, the treatment option data T′ may be provided via a communications or control interface CI to the medical device D. The medical device D may be controlled, based on the treatment data T, to, for example, treat the patient, or to initiate one or more steps in relation to the patient's future treatment. The treatment device may include a radiation treatment device such as linac, a contrast agent pump, a surgical robot, etc.

The system SYS for predicting treatment recommendations T may be implemented by one or more computing devices PU. For example, in one embodiment a Cloud based architecture is used where the system SYS is implemented by one or more servers, preferably working in concert. The system SYS may provide via a suitable communication channel the computed treatment options to a terminal (user) device. In other embodiments the system SYS is implemented fully or in parts on the terminal device or other edge device of a communications network. The terminal device may be a desktop computer, a laptop, tablet, smart phone, smart watch, or other computing device of a clinical user or indeed of the patient itself. Preferably, but not necessarily, the computing device PU on which the system is implemented is powered by one or more processors capable of parallel processing. For example, processors of multi-core design may be used to parallelize the computing performed by the predictor module PM.

In embodiments, the output treatment option T may be a specific treatment regime, such as medication regime or radiation treatment plan with specific parameters. However, more preferably and in other embodiments, the treatment option data T as computed by the predictor module PM may define instead a certain treatment template or treatment option type that does not generally provide detailed parameters, but provides instead a set of ranges or types of treatment parameters that may lead to a favorable outcome for the patient given the biodynamical measurements m received. For example, in radiation therapy which is envisaged as one treatment option herein, such as external or internal radiation beam therapy, the treatment option T may relate to a certain type of radiation profiles in respect of an organ of interest and/or organs at risk, or to a certain type of fluence maps, etc.

The predictor module PM may be implemented in some embodiments by explicit, analytical modelling techniques, where the relationship between the measurements m and the treatment options T are modelled by explicit function(s). For example, the predictor module PM may be implemented in some embodiments by a look-up table which tables types of measurements versus associated treatment options.

However, preferably, the predictor module is implemented in machine learning (“ML”) by using one or more trained machine learning models M. Machine learning allows harvesting historical medical knowledge that resides in prior medical data amassed from cohorts of patients in historical examinations or cases over time in the past. The prior medical data may be found in extant medical databases MD', such as data sets comprising medical records of prior patients and/or indeed prior records of the give patient PAT.

The lower left hand part of FIG. 1 illustrates such medical data set as plural medical records j held in a database MD'. The medical records j for a cohort of patients (index j refers to different patients) may include biodynamical measurements m/acquired in the past as part of diagnostics/medical fact finding, associated treatment options Tj for patient j, and possibly outcome data Oⱼ. For different patients j,j′ there may be different treatments Tⱼ,T_{j′} yielding different outcomes Oⱼ,O_{j′}. Part of the reason for such different treatment options and may be grounded in different medical aspects such as different patient histories, different patient physiologies, different genetic make-up of patients, etc. Each of the said foregoing medical aspects may account for different patients having responded differently to different treatment options.

It may be assumed that it is was the then treating medical practitioners who, using their medical knowledge, decided the respective treatment option that was deemed suitable for the patients in the cohort as recorded in the historical data records j of medical databases MD′. Thus, the collection of historical medical records MD′ may be thought of encoding this medical knowledge. In particular, there may be a latent relationship [symbol] between, on the one hand, the medical measurements mⱼ collected from the patients in the past, and the associated treatment options Tⱼ, and optionally their associated outcomes Oⱼ. It may be difficult, or outright impossible, to model this latent relationship [symbol] , referred to herein as the measurement-versus-treatment option (“MvT”)-relationship, analytically in terms of analytical function of a set type. Machine learning offers a practical way forward, allowing a more flexible modelling approach where one is not tied to a specific analytic functional expression of this relationship from the outset. ML may use the prior medical data collectively to implicitly model for the latent MvT relationship.

It is proposed herein in preferred embodiments to use any one of a number of different machine learning models to learn this relationship [symbol] from the said prior medical data MD′. Upon sufficient training based on a suitably large and representative such prior data MDA from a varied cohort of prior patients, the predictor module PM can be constructed in a way that allows reliable treatment option recommendations/predictions be computed for a given patient, based on current, or previously acquired, biodynamical measurement for the given patient PAT at hand. The systems SYS allows reducing overall health care costs by computing in a computational efficient manner suitable treatment options that are personalized to the respective patient. The assumption being here that the different latent parameters that determine whether or not a treatment option is suitable for a given patient (that is, has a positive or favorable outcome) is implicitly encoded in the totality of biodynamical measurements that are available for a given patient.

The biodynamical measurements represent an over-time aspect of the constitution of the patient and may be viewed as a tell-tale for the way he or she may respond to a treatment option. The biodynamical measurements as used herein may be stored or provided as time series data or spatially varying data, depending on the senor type used for their acquisition. Some measurements may vary over time and space. For example, biodynamical measurements may include over time/measurements m m, acquired by sensor(s) S of the medical measurement device MD, such as an ECG apparatus, an EEG apparatus, laboratory equipment that establishes over time blood parameters, or transmission/emission imagery acquired of the patient. Some medical devices MD work by having the sensor(s) S pick-up the signal of interest, such as electrodes attached to the patient's skin for acquisition of EEG or ECG data.

In other embodiments the medical measuring device MD includes a signal source SS which generates an interrogator signal to which the patient is exposed to. The interrogator signal interacts with patient tissue, and produces in response thereto, a response signal which is then picked up by the sensor S. Such is the case for example for medical imaging data acquired over time, which is one example for time and spatially varying data, in turn an example of biodynamical measurements envisaged herein. For example, in a CT or C-arm setting, during imaging session, an X-ray source as signal source SS rotates around the examination region with the patient in it to acquire projection imagery from different directions, and, preferably, over a period of time. The projection imagery is detected by an X-ray sensitive detector S. The X-ray sensitive detector S may rotate opposite the examination region with the X-ray source SS, although such co-rotation is not necessarily required, such as in CT scanners of 4^{th} or higher generation. The signal source SS, such an X-ray source (such as an X-ray tube), is activated so that an X-ray beam issues forth from a focal spot in the tube during rotation. The X-ray beam traverses the examination region and the patient tissue therein, and interacts with same to so cause modified radiation to be generated due to, for example, differential attenuation and scattering effects, etc. The modified radiation is detected by the X-ray sensitive detector S as intensities for example. The X-ray sensitive detector S is coupled to acquisition circuitry such as DAQ to capture the projection imagery in digital form as digital imagery. The same principles apply in (planar) radiography, only that there is no rotation of source SS during imaging. In such a radiographic setting, it is this projection imagery that may then be examined by the radiologist. In the tomographic/rotational setting, the muti-directional projection imagery is processed first by a reconstruction algorithm that transforms projection imagery from projection domain into sectional imagery in image domain. Image domain is located in the examination region. C-arm imagers may be used to acquire over time projection imagery of a region of interest, such as the human heart. Bi/-multi-plane C-arm scanner allow multi-directional imaging without rotation during imaging. Fluoroscopy, in particular angiography, imaging is envisaged herein in preferred embodiments as will be explored below in greater detail with reference to some examples.

Imagery thus acquired are one example of biodynamical measurements. However, such imagery may not necessarily result from X-ray imaging. Other imaging modalities, such as emission imaging, as opposed to the previously mentioned transmission imaging modalities, are also envisaged herein such as SPECT or PET, etc. In addition, magnetic resonance imaging (MRI) is also envisaged herein in some embodiments, and so is optical coherence tomography, and others still.

In MRI embodiments, the said signal source SS is formed by radio frequency coils which may also function as detector sensor(s) S, configured to receive, in receive mode, radio frequency response signals emitted by the patient residing in a magnetic field. Such response signals are generated in response to previous RF signals transmitted by the coils in transmit mode. There may dedicated transmit and receive coils however in some embodiments instead of the same coils being used in the said different modes.

In emission imaging, the source SS is within the patient in the form of a previously administered radio tracer which emits radioactive radiation that interacts with patient tissue. This interaction results in gamma signals that are detected by as suitably configured sensor S, in this case gamma cameras, arranged preferably in an annulus around the examination region where the patient resides during imaging.

Instead of, or in addition to the above mentioned modalities, ultrasound (US) is also envisaged, with signal source SS and sensor S being suitable acoustical US transducers.

As noted earlier, some imaging equipment may provide biodynamical measurements that may vary in both, space and time. For example, over time imagery (such as projection angiograms, computed tomography angiograms (“CTA”), and others) represents spatially and time varying data, envisaged herein in some examples, such as medical imagery to capture motion of cardiac vessels during one or more cardiac cycles.

It may be preferred herein in some embodiments to combine biodynamical measurements form different sensor types of acquisition equipment into “super”-biodynamical measurements for better results. Thus, for example, ECG data and medical imagery, may be provided for joint processing by the predictor model PM. The underlying ML model M for such module PM may be trained jointly on such data.

In embodiments, machine learning proceeds in general in two/three phases: a training phase, an optional testing phase, and a deployment/inference phase.

In training phase, based on the prior training data, a model M is adapted. The training phase may be a one-off operation, or may be repeated as more training data becomes available. After training phase, there may be an optional testing phase, and after testing phase, a deployment/inference phase. Thus a trained model may be used in testing or deployment.

In embodiments, model adaptation in training phase may include implicit or explicit modelling. For example, neural network type models may be used as an example for explicit ML modelling, whilst data clustering approaches (also envisaged herein) represent examples of implicit ML modelling.

In explicit modelling, in the prior training phase, parameters of the model M are adapted based on the training data and according to pre-defined criteria as measured by one or more objective functions for example.

Once the model is sufficiently trained, which may be established by objective criteria, such as the behaviour of the objective function, the model may be used for testing or deployment in day-to-day medical practice. Thus, in explicit modelling, a (preferably large) set of parameterized functions may be used to model the relationship [symbol] . The parameters are adapted based on the training data. Such modelling may include neural network type modelling, support vector machines, or others. In other types of ML, such as the said implicit modelling, it is the training data set itself that represents the model, optionally including a set of descriptors as a way to structure the data for use in deployment. One such type of implicit modelling includes the said clustering type machine learning algorithms, specifically envisaged herein in embodiments. Clustering type algorithms are preferred herein because the sometimes inherent “fuzziness” of the treatment options can be better represented graphically as clusters which are shown against the current measurements m of the patient. Treatment options have been found to not always lend itself well to hard classifications that tend to pin the treatment options down too much. Clustering better captures fluidities or tendencies across and over such hard classifications. It can be established by user more easily, more intuitively, into which category or cluster the patient tends to fall. Each cluster is thought to be associated with a certain type or template of treatment option type (optionally with associated outcome data) rather than with a hard set of pre-defined parameters. The medical user can thus more easily ascertain into which kind of “treatment option region” the patient is more likely to experience a positive treatment outcome, without being pressed into hard choices down to parameter level.

An example of output data generatable by the predictor module PM, in particular when implemented based on a cluster type algorithm, is shown in FIG. 2. FIG. 2 at the same time is an illustration of an embodiment of a graphic display GD as may be generated by the graphics display generator GDG that processes the output data received by the predictor module PM. For example, in the said clustering type algorithms, after training, the predictor module PM provides an indication, such as centroid or other descriptor, for one or more of the given pre-defined cluster(s) Cᵢ into which the patient is likely to fall into. Each cluster represents one (or more related) (different) treatment option(s) Tᵢ. For example, and as illustrated in FIG. 2, the clusters Cᵢ may be generated as point clouds in a vector space.

Each point corresponds to a set of parameters represented in the suitably high dimensioned vector space. Suitably encodings may include, one-hot-encodings, embedding or others. A measurement preprocessor PP processes raw measurements into a suitable representation, measurement preprocessor PP may perform mapping, scaling, or normalization operations as required. Preprocessor PP may use ML in its own right to obtain a suitable data representation for fast and efficient processing by module PM. In more detail, the preprocessor PP may obtain suitable encodings by for example simply mapping to a list or symbols or by ML techniques such as mapping to a latent space of an autoencoder AE) network, or variational AE, or others. Whilst the vector space may be high dimensional with dimension higher than 3, a 3D or 2D (as shown in FIG. 2) graphical representation may still be obtained by dimensional reduction techniques, such a PCA, or other. The said parameters that span the vector space may include suitably coded vectors for the measurements, and optionally enriched with patient contextual information K, such as bio-characteristics (weight, age, sex, etc), patient history, etc. In some preferred embodiments, coronary motion patterns as one example of biodynamic measurements. The contextual information k parameters may further include and one or more of baseline clinical demographics, clinical characteristics of the patients, laboratory values, medications, procedural and follow-up data coronary motion pattern.

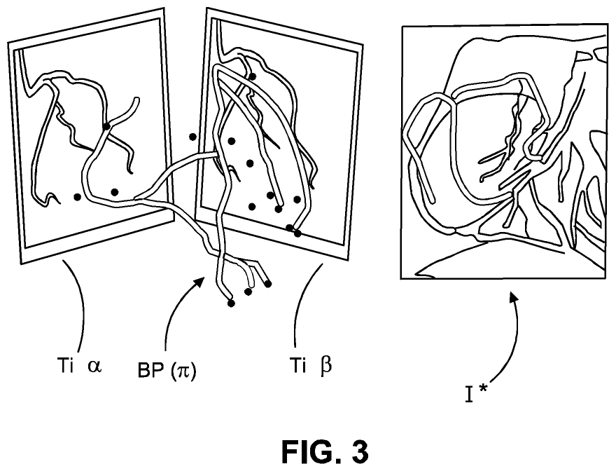

The nature of the preprocessor PP may depend on the clinical domain and nature of the measurement. A detailed example based on cardiac vessel image data, with suitable data preparation operation by preprocessor PP to capture vessel motion pattern as one example of biodynamical measurements m, will be described below at FIG. 3.

With continued reference to FIG. 2, some or each of the point clouds Cᵢ may be associated with a graphical or textual description that provides an indication of the characteristics of the treatment option that is represented by the respective cluster Cᵢ.

The similarity or proximity to the given clusters is computed by the predictor module upon processing the medical measurements of the current patient. A graphical widget or indicator. illustrated as an example cross (“X”) in FIG. 2, may be overlaid on the point cloud representations of the clusters so that the user can discern into which treatment type category (or indeed plural categories) the given patient tends to falls. For example, in the embodiment in FIG. 2, it is the upper left hand side cluster into which the current patient X is likely to fall and could benefit from the associated treatment option at the associated treatment outcome. It may well be that the patient PAT falls into multiple clusters Cᵢ in which case the indicator X computed by module PM is rendered in between a group of clusters. FIG. 3 shows one embodiment of biodynamical measurements as may be used in embodiments envisaged herein. The biodynamical measurements m may include X-ray imagery, or imagery of other types of medical modalities, acquired of cardiac vessels, such as coronary veins or arteries of a patient. Contrast agent is preferably administered prior to acquisition to improve contrast. In embodiments, at least two X-ray projection images π_{α}, π_{β} are obtained along different projection directions α,β. A back-projection operation BP is used to obtain, based on projections π_{α}, π_{β}, a representation in 3D space (image domain) of the intensity distribution as recorded by measurements π_{α}, π_{β}of the region of interest (such as the cardiac vessels). Cross-sectional (tomographic) imagery I* (illustrated on the right-hand side of FIG. 3) or other renditions may be obtained from the back-projected data. By using a larger number of projections, reconstruction I* may be refined. However, using merely two projections as illustrated may be sufficient in some embodiments.

In more detail, and according to some embodiments, the measurements m may include motion pattern descriptive vector of a 4D vector field representing the motion of the coronary arteries during one cardiac cycle. ML can be used in preprocessor PP, trained for example based on a, preferably convolutional, neural network to segment for coronaries from at least two angiographic X-ray sequences π_{α}′, π_{β}′, with index t indicating acquisition time. Alternatively, the preprocessor PP may use non-ML techniques, or ML approaches other than NN is used. The segmentations may be back-projected by preprocessor PP into 3D space as mentioned above and as illustrated in FIG. 3. The over-time CTA data with segmented coronary structures can serve as ground truth. Optionally, but preferably, ECG data recorded alongside with the angiograms π_{α}′, π_{β}′ may be used to form super-measurements that may be processed jointly by predictor PM for better robustness, as the ECG data is indicative of the cardiac phase over time, and may be used together with contrasted angiograms π_{α}′, π_{β}′ to select a frame (for projection α, β, or both) in the angiographic sequences, representative of the associated cardiac phase. Such selection may be done automatically (e.g. based on contrast filling or least overlap of vessels) or manually, or in any other clinically meaningful way. The two (or more) angiographic sequences π_{α}!, π_{β}′ are preferably taken from different angles (e.g. at least 30 degrees apart). Imaging parameters such as C-arm angiography system parameters (including for example rotation and angulation angles, field of view, source image distance, etc) used for the acquisition of π_{α}′, π_{β}′ may be used to perform the (spatial perspective) back-projections m=I* ′ over time.

The above-described procedure above can be performed for multiple phases in the cardiac cycle (e.g. 15 phases). The individual back-projections m=I* ′ then form a 4D sequence (3 spatial dimensions over time). This sequence I* ′could directly be used as the above-mentioned descriptive vector. Alternatively, and optionally, a spatial registration step between the individual phases (e.g. using the iterative closest point algorithm, or other) could be used to establish a 4D vector field, which would then be the descriptive vector to serve as prepared measurements m.

Alternatively to the procedure above, the measurements m may be prepared by preprocessor PP as a 2D+t motion field, preferably over at least one cardia cycle for at least two projection directions α,β. The 2D+t motion field can be calculated for some or each of the angiograms at times t, π_{α}′, π_{β}′, by image registration of plural, such as all, frames in the sequence, relative to a pre-selected reference frame, for example the frame with most contrast filling. The descriptive vector m then comprises a table of these 2D+t motion fields, optionally together with their system parameters (rotation and angulation angles, field of view, source image distance, etc.). Optionally still, the 2D+t motion fields are constrained to a single cardiac cycle. The preprocessor stage PP may be used during training to suitable prepare the training data, and/or during deployment to prepare the incoming new measurements encountered in deployment or testing.

The above described example of CTA based cardiac vessel motion is not limiting herein. For example, other vessels, indeed other regions of interests not related to the heart may be imaged instead. In addition or instead, tomographic imaging, whilst preferred, is not required herein. Purely projection based over-time imaging is also envisaged. Imaging modalities other than X-ray based may be used instead, such as MRI, SPECT/PET, US, etc.

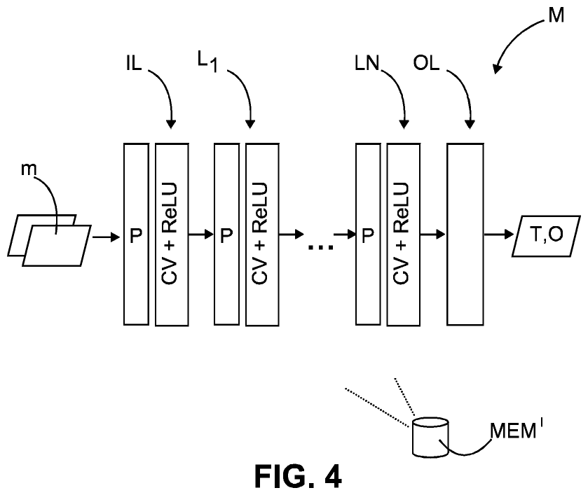

Reference is now made to FIG. 4 which shows a machine learning model M of the artificial neural network (“NN”) type according to one embodiment for explicit modelling. Preferably deep learning is used, where the neural network model includes more than one hidden layer Lj between input layer IL and output layer OL.

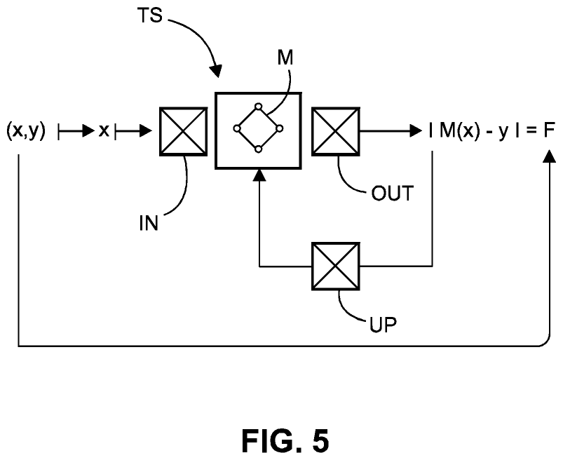

The model M may be trained by a computerized training systems TS to be described more fully below at FIG. 5. In training, the training system TS adapts an initial set of (model) parameters θ of the model M. In the context of neural network models, the parameters are sometime referred to herein as weights.

The machine learning model M may be stored in one (or more) computer memories MEM′. The pre-trained model M may be deployed as a machine learning component that may be run on a computing device PU, such as a desktop computer, a workstation, a laptop, etc or plural such devices in a distributed computing architecture. Preferably, to achieve good throughput, the computing device PU includes one or more processors (CPU) that support parallel computing, such as those of multi-core design. In one embodiment, GPU(s) (graphical processing units) are used, in particular (but not only) when the model is of an artificial neural network type, an embodiment which is now explained in more detail.

Specifically, and referring now in more detail to FIG. 4, this shows a convolutional neural network M in a feed-forward architecture, as one example of an NN. The network M comprises a plurality of computational nodes arranged in layers IL, Lⱼ. OL in a cascaded fashion, with data flow proceeding from left to right and thus from layer to layer. Recurrent networks are not excluded herein, in particular when dealing with time data, such as the biodynamical measurements. Convolutional networks have been found to yield good result when processing image data. The network may be convolutional and of recurrent type, in some preferred embodiments.

In deployment, the input data, including measurements m are applied to input layer IL, optionally complemented with contextual data K. The input data m then propagates through the sequence of hidden layers L₁-L_{N} (only two are shown, but there may be merely one or more than two), to then emerge at an output layer OL as an estimate output M(m)=T, which represents the associated treatment option T.

The layers of the network, and indeed the input m and/or output T, and the input and output between hidden layers (referred to herein as feature maps), can be represented as vectors or two or higher dimensional matrices (“tensors”) for computational and memory allocation efficiency.

Preferably, some or all of the layers are convolutional layers, that is, they include one or more convolutional filters CV which process an input feature map from an earlier layer into intermediate output, sometimes referred to as logits. An optional bias term may be applied by addition for example. An activation layer processes in a non-linear manner the logits into a next generation feature map which is then output and passed as input to the next layer, and so forth. The activation layer may be implemented as a rectified linear unit RELU as shown, or as a soft-max-function, a sigmoid-function, tanh-function or any other suitable non-linear function. Optionally, there may be other functional layers such as pooling layers P or drop-out layers to foster more robust learning. The pooling layers reduce dimension of output whilst drop-out layer sever connections between node from different layers.

In embodiments, downstream of the sequence of convolutional layers there is output layer OL which is configured for a classification or regression task. In particular a classification result may be sought, such as in classification for a treatment option T from a set of predefined treatment templates Alternatively, output layer is configured to regress measurement m into treatment option T type, such as in vector or matrix/tensor form, depending on the clinical context. For example, in RT therapy, the treatment option T may represent a particular fluence maps, or metrics characterizing such fluence maps, or treatment plan templates more generally. Downstream or upstream a run of convolutional layers, there may be one or more fully connected layers. When configured for classification tasks, output layer OL may be implemented as a soft-max layer for example.

It will be understood that the above-described model M in FIG. 4 is merely according to one embodiment and is not limiting to the present disclosure. Other neural network architectures are also envisaged herein with more or less or different functionalities than described herein, such as pooling layers or drop-out layers or others still. What is more, models M envisaged herein are not necessarily of the neural network type at all. Other, statistical regression methods based on sampling from training data are also envisaged herein in alternative embodiments. Still other techniques may include Bayesian networks, or random fields, such as Markov type random field and others.

FIG. 5 shows an embodiment of a training system TS as may be used to train the machine learning model M, such as in explicit or implicit modelling. The system TS may include an updater UP hat updates/adapts the model parameters for training data set structure.

Training is the process of adapting parameters of the model M based on the training data. An explicit model is not necessarily required as in some examples it is the training data itself that constitutes the model, such as in clustering techniques or k-nearest neighbors, etc, mentioned above.

In explicit modelling, such as in NN-based approaches and many others, the model may include as system of model functions/computational nodes, with their input and/or output it least partially interconnected. The model functions or nodes are associated with parameters θ which are adapted in training. The model functions may include the convolution operators and/or weights of the non-linear units such as a RELU, mentioned above at FIG. 4 in connection with NN-type models. In the NN-case, the parameters θ may include the weights of convolution kernels of operators CV and, optionally, of the non-linear units. The parameterized model may be formally written as M^{θ}. The parameter adaptation may be implemented by a numerical optimization procedure. The optimization may be iterative. An objective function/may be used to guide or control the optimization procedure. The parameters are adapted or updated so that the objective function is improved. The input training data x=m′ₖ is applied to the model. k is an index that runs over pairs of training data in a supervised setting. The model M responds to produce training data output M^{θ}(m′ₖ)=T′ₖ. The objective function is a mapping from parameters space into a set of numbers. The objective function/may measure a combined deviation between the training data outputs M^{θ}(m′ₖ) and the respective targets y=T′ₖ. Parameters are iteratively adjusted to that the combined deviation decreases until a user or designer pre-set stopping condition is satisfied. The objective function may use a distance/similarity measure ||. || to quantify the deviation.

In some embodiments, but not all, the combined deviation may be implemented as a sum over some or all residues based on the training data instances/pairs (m′_{k′}T′ₖ)^{k}, and the optimization problem in terms of the objective function may be formulated as:

$$arg {min}_{θ} F = {\sum }_{k} ‖ {M}^{θ} ( {m}_{k}^{′} ) , {T}_{k}^{′} ‖ \qquad ( 1 )$$

In the setup (1), the optimization is formulated as a minimization of a cost function F, but this is not limiting herein, as the dual formulation of maximizing a utility function may be used instead. Summation is over training data instances k.

The cost function/may be pixel/voxel-based, such as the L1, or the smoothed L1-norm, L2-norm, Hubert function, or the Soft Margin cost function. For example in least squares or similar approaches, the (squared) Euclidean-distance-type cost function F in (1) may be used for the above mentioned regression task for regression into treatment options T. When configuring the model M as a classifier that classifies into treatment options from among a set of such options, the summation in (1) may be formulated instead as one of cross-entropy or Negative Log Likelihood (NLL) divergence or similar.

The exact functional composition of the updater UP depends on the optimization procedure implemented. For example, an optimization scheme such as backward/forward propagation or other gradient based methods may then be used to adapt the parameters θ of the model M so as to decrease the combined residue for all or a subset (m′ₖ, T′ₖ) of training pairs from the full training data set. Such subsets are sometime referred to as batches, and the optimization may proceed batchwise until all of the training data set is exhausted, or until a predefined number of training data instances have been processed.

Reference is now made to equation (2) below, which represents an example of the earlier described implicit type modelling approach as may be used in training system TS, such as in clustering or similar ML approaches:

$$arg {min}_{θ = \{ Ck \}} F ( C , {m}^{′} ) \qquad ( 2 )$$

In such models, it is the training data itself that represents the model, optionally enhanced by descriptors δ of the training data τ, such as centroids, etc or other structuring mechanisms. The descriptors may be computed based on the training data and a suitable similarity measure Σ defined for elements of the training data set. For example, k-means clustering, or similar algorithms may be used. Eq. (2) or (1) may be implemented over one or more iterations. Objective function/may be formulated in terms of distance function Σ that quantifies a distance between training data measurement m′, and existing descriptors δ for clusters computed in previous iteration cycles.

In clustering type algorithms, objective function F is improved iteratively for some or each m′ (a sample from the training data), and optimization is over subsets Cₖ of the training data. F in general corresponds to an underlying similarity Σ or distance measure. For each m′ in the current training data set, either a new class m′∈Cₖ is opened, or m′ is deemed a member of class C_{k′} opened in an earlier iteration cycle k′<k. Thus, in such implicit schemes, adapting the model includes structuring the data set, for example by finding a set of subsets that define the clusters in the training data set. Once clustering of the current data set (the learning phase) concludes, a newly received measurement in testing or deployment may be assigned to one (or more) classes that according to F or Σ, as the case may be.

Clustering or similar implicit ML modelling approaches are in general unsupervised, so there is no pairing of the training data into training input versus target required as in some of the above-described explicit ML modelling approaches. The similarity measure Σ may be based on Euclidean squared distance, for example, or may be based on others. In such or other embodiments it may be is assumed that the biodynamical measurements are mapped as elements of a suitable vector space, so that the similarity measure can be well-defined in order to compute the descriptors for the clusters in the learning step. One example of such mapping are descriptive vectors, such as described above in FIG. 3.

At the conclusion of training, the training data is clustered into clusters Cᵢ, optionally with associated descriptors δᵢ. A new measurement m is received, either in testing or deployment phase. The new measurement m is applied to the descriptors to find the best fitting one or more descriptors (according to F), and hence their associated clusters Cᵢ into which the measurement m′ is deemed to fall. The respective treatment(s) Tᵢ associated with the said one or more clusters so identified may then be used to inform further medical steps for the given patient.

Training, whether using implicit or explicit modelling, may be a one-off operation, or may be repeated once new training data become available.

In more detail and with reference to the example in FIG. 3, when the clustering is driven by a similarity measure as mentioned above, a grouping of similar motion patterns is preferably effected, such as for the cardiac vessel motions mentioned above. A suitable similarity measure between two given motion vector fields could be configured by first spatially scaling the 4D motion field such that a bounding box for the region of interest (eg, the heart) is the same, and then normalizing the vector field by the average based on vector magnitudes. The integral of dot-products of the normalized vectors may be used to represent similarity between the vector fields.

This clustering may be performed by the training system TS for plural, such as all, patients data items in the training data set.

Alternatively to being similarity measure driven, the clustering by system TS can be performed by first clustering the patient data on one or more selected or pre-defined contextual parameters κ (e.g. mortality, complications, outcome on an appropriate scale, etc.), and then train the ML model, such as a convolutional network, to match the descriptive vectors m with the predefined clusters. After training, during testing or deployment, a (new) coronary motion descriptive vector m is established by data preprocessor PP for a given patient who is currently being treated. Then a lookup in the pre-clustered data is performed by predictor module PM in order to ascertain into which cluster the current patient falls. Medical information for that cluster can be displayed on display device DD for clinical staff. The data can be used to determine an (optimal) treatment strategy, perform device selection, establish a follow-up plan and/or risk monitoring plan, etc.

Optionally, segmented structures, such as the mentioned 4D coronary arteries, that can be extracted as described above or similar, may be used for overlaying and roadmap purposes.

Instead of using motion descriptors of coronary arteries as biodynamical measurements m herein, other descriptive features can be used instead or in conjunction to the above as biodynamical measurements. This may be in the same clinical domain or other clinical domains. Examples envisaged herein include herein cerebral stroke, where the descriptive vector m includes brain perfusion or oxygen consumption, or for peripheral artery disease, where the descriptive vector m includes blood perfusion, etc.

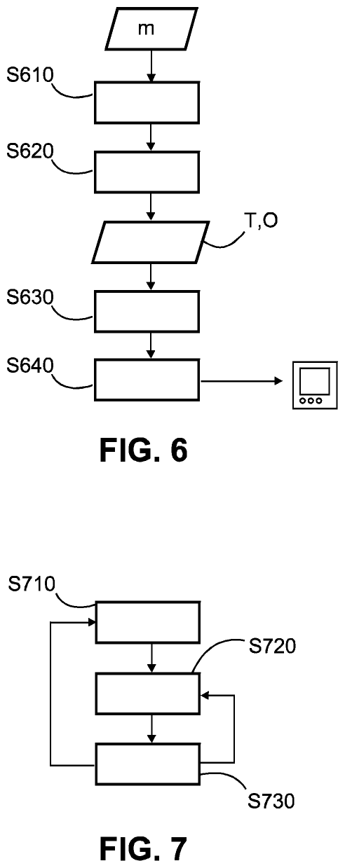

Reference is now made to FIG. 6 which shows a flow chart of a method for predicting treatment options and/or associated outcome data based on biodynamical measurements m for a patient. It will be understood that the steps described below are not necessarily tied to the architecture described above, but may be understood instead as a teaching in their own right, optional outcome data, me also be suitably prepared by processor PP in vector form for example, and may relate to time periods that indicate for how long the patient was symptom free post treatment. Any other medically recognized outcome metric may be used instead.

At step S610 one or more biodynamical measurements m for a given patient are received.

At step S620 treatment option data T is computed based on the measurement m. This computing step may be based on a trained machine learning model as described above and as applied to the measurement m. Thus, the method may be used during testing or deployment after training. Optionally, the output data includes outcome data o associated with the computed treatment option T.

The output data T may include an indication of a cluster in a pre-defined space of clusters, the said clusters previously derived from a training data set. Thus, the machine learning model may be one of a cluster type model, such as k-means clustering, or others, such as mixture models or EM-type algorithms more generally, or PCA, etc. The indication indicates one or more of the clusters into which the patient, given the measurement m, is likely to fall. Thus, the patient is deemed to benefit from treatment options pre-associated with the respective cluster. In other ML embodiments, (kernel) support vector machine types algorithms may be used, or K-nearest neighbors, decision trees, or any other ML classification scheme.

In ML approaches other than clustering, the output data computed at step S620 may be provided as a vector. Entries of the vector may code for respective treatment option types. Matrices may be used instead for representation.

At step S630 the treatment data and, optionally the associated outcome data, is provided at step S630 for displaying, storing or otherwise processing.

At step S640 the output data T, o may be visualized in a graphics display on a display device. The graphics display may include a graphical representation such illustrated in FIG. 3 of clusters, wherein each cluster represents a specific treatment option. Outcome data may be data associated with each such cluster, such a pop-up text field or other. Details of the treatment option ma likewise be presented as text and/or graphically upon user interaction through a suitable user interface, such as a GUI, or other. A text or graphic widgets may emerge when the user designates the indicated cluster (see “X” in FIG. 2). Designation may be by a pointer tool, such as computer mouse, stylus or via touch screen action.

The outcome data as predicted herein in step S640 may include thus indications of treatment options which may be suitable for a patient, either at the point of time when the prediction is made, or at some point of time in the feature. Alternatively, it is already established that the patient is in need for treatment for a certain disease or conditions, and the clusters represent treatment options for this specific disease or condition. Thus, in the first case, the treatment option(s) predicted may relate to a disease condition that was not a priori known prior to the prediction. Thus, the proposed method and system may be used in a focused sense to find treatment options for a given disease, or in a more global, exploratory sense to find treatments for disease/conditions from which the patient, given their measurements m, may benefit at some point in time in the future.

Reference is now made to the flow chart of FIG. 7 which shows a method of training, explicitly or implicitly, a machine learning model as may be used in the method according to FIG. 6.

At step S710, previous medical data, including biodynamical measurements m′ of the current or other patients, are accessed, procured, or otherwise sourced. The data so accessed, procured or sourced represents training data. The training data may be accessed, sourced, procured or otherwise obtained by querying historic data sets, such as health data records from prior patients and/or from previous records for a given patient. Preferably it is assured that a data from a suitable large and varied cohort of patients is queried to ensure a suitably representative set of such training data.

At step S720 a machine learning model is adapted based on the training data. The said adaptation may also be referred to herein as training of the ML model. The adaptation may be implicit. such as in clustering, or may be explicit such as in neural network type modelling or other. The adaptation may proceed in an iterative manner. The adaptation may be driven by an objective function F( ), such as cost function, to improve objective function. Parameters of the model may be adapted and/or the training data set may be structured.

At step S730, after training S720, the suitably trained model is then provided for testing or deployment. In testing, the trained model is supplied to some of the training data not used during the training, but set aside beforehand, in order to supply this data as input to the training model to assess performance of the model.

In deployment, the model may be used by clinical staff in day-to-day clinical practice by applying new data (biodynamical measurement(s)) m, not drawn from the training/test data. The new data m may be retrieved from storage in an offline embodiment, or may be currently acquired by measurement device M as such measurement(s) m for a given patient in on-line embodiments. The new data m is applied to the trained model to compute an associated treatment option and/or outcome data for the given patient, as described above.

The components of system SYS may be implemented as one or more software modules, run on one or more general-purpose processing units PU such as a workstation associated with the imager IA, or on a server computer associated with a group of imagers.

Alternatively, some or all components of system SYS may be arranged in hardware such as a suitably programmed microcontroller or microprocessor, such an FPGA (field-programmable-gate-array) or as a hardwired IC chip, an application specific integrated circuitry (ASIC). In a further embodiment still, the system SYS may be implemented in both, partly in software and partly in hardware.

The different components of the system SYS may be implemented on a single data processing unit PU. Alternatively, some or more components are implemented on different processing units PU, possibly remotely arranged in a distributed architecture and connectable in a suitable communication network such as in a cloud setting or client-server setup, etc. For example, the trained module PM may be run in on computing system, whilst the training system TS is run on another. The preprocessor PP may be implemented on yet another computing system. Instead, any two or all tree of module PM, preprocessor PP and training system TS may be implemented on a single computing system PU.

One or more features described herein can be configured or implemented as or with circuitry encoded within a computer-readable medium, and/or combinations thereof. Circuitry may include discrete and/or integrated circuitry, a system-on-a-chip (SOC), and combinations thereof, a machine, a computer system, a processor and memory, a computer program.

In another exemplary embodiment of the present invention, a computer program or a computer program clement is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above-described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it which computer program clement is described by the preceding section.

A computer program may be stored and/or distributed on a suitable medium (in particular, but not necessarily, a non-transitory medium), such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope. Such reference signs may be comprised of numbers, of letters or any of alphanumeric combination.

1. A system for predicting a patient treatment option, the system comprising:
a processor configured to:
receive input data including biodynamical measurements in respect of a patient;
process the biodynamical measurements to obtain output data including an indication for a treatment option for the patient,
wherein the biodynamical measurements are processed based on a trained machine learning model, previously trained on patient data from a cohort of patients, and
wherein the machine learning model is implemented based on a clustering algorithm.

2. The system of claim 1, wherein the biodynamical measurements is a time series.

3. The system of claim 1, wherein different treatment options correspond to different clusters, and wherein the indication includes an indication of one or more of the different clusters.

4. The system of claim 3, wherein the processor is further configured to generate a graphics display for display on a display device, the graphics display provides a visualization of the indication.

5. The system of claim 4, wherein the graphics display includes a visualization of the different clusters and a graphical indicator in respect of the patient indicative of proximity or similarity to the the different clusters.

6. The system of claim 1, wherein the biodynamical measurements incudes one or more of: coronary vessel motion data, perfusion data, electrocardiogram data, electroencephalogram data, and oxygenation data.

7. The system of claim 1, wherein the biodynamical measurements include image data.

8. The system of claim 1, wherein the output data includes outcome data for the treatment option.

9. A training system for training, based on the training data, the machine learning model of the system of claim 1.

10. A computer-implemented method for predicting a patient treatment option, the method comprising:
receiving input data including biodynamical measurements in respect of a patient; and
processing the biodynamical measurements to obtain output data including an indication for a treatment option for the patient,
wherein the processing is based on a clustering algorithm.

11. The computer-implemented method of claim 10, wherein the biodynamical measurements are processed based on a trained machine learning model previously trained on patient data from a cohort of patients, and wherein the machine learning model is implemented based on the clustering algorithm.

12 A non-transitory computer-readable storage medium having stored a computer program comprising instructions, which, when executed by a processor, cause the processor to:
receive input data including biodynamical measurements in respect of a patient; and
process the biodynamical measurements to obtain output data including an indication for a treatment option for the patient,
wherein the biodynamical measurements are processed based on a trained machine learning model previously trained on patient data from a cohort of patients, and wherein the machine learning model is implemented based on a clustering algorithm.

13. (canceled)

14. The method of claim 10, wherein the biodynamical measurements is a time series.

15. The method of claim 10, wherein different treatment options correspond to different clusters, and wherein the indication includes an indication of one or more of the different clusters.

16. The method of claim 15, further comprising generating a graphics display that provides a visualization of the indication.

17. The method of claim 16, wherein the graphics display includes a visualization of the different clusters and a graphical indicator in respect of the patient indicative of proximity or similarity to the the different clusters.

18. The non-transitory computer-readable storage medium of claim 12, wherein the biodynamical measurements is a time series.

19. The non-transitory computer-readable storage medium of claim 12, wherein different treatment options correspond to different clusters, and wherein the indication includes an indication of one or more of the different clusters.

20. The non-transitory computer-readable storage medium of claim 19, further comprising generating a graphics display that provides a visualization of the indication.

21. The non-transitory computer-readable storage medium of claim 20, wherein the graphics display includes a visualization of the different clusters and a graphical indicator in respect of the patient indicative of proximity or similarity to the the different clusters.
